(19) United States
(12) Patent Application Publication
(10) Pub. No.: US 2026/0286441 A1
(43) Pub. Date: Sep. 24, 2026

(54) METHODS OF STRATIFYING A SUBJECT BY EVALUATING TELOMERE LENGTH

(71) Applicant: The Johns Hopkins University, Baltimore, MD (US)

(72) Inventor: Mary Y. Armanios, Ellicott City, MD (US)

(21) Appl. No.: 19/168,398

(22) PCT Filed: Mar. 28, 2024
(86) PCT No.: PCT/US2024/022061

§ 371 Date: Sep. 24, 2025

Related U.S. Application Data
(60) Provisional application No. 63/455,400, filed on Mar. 29, 2023.

(51) Int. Cl. C12Q 1/6886

(52) U.S. Cl. CPC C12Q 1/6886; C12Q 2600/118; C12Q 2600/156

(57) ABSTRACT

Provided herein are methods of treating pulmonary fibrosis or liver disease or bone marrow failure or primary immunodeficiency in a subject having short telomere syndrome (STS), the method comprising: (a) detecting the presence of STS in the subject; and (b) administering a therapy, wherein the therapy does not suppress T cell immunity or wherein the therapy does not lower immunosuppression.

### CROSS-REFERENCE TO RELATED APPLICATIONS

This application claims the benefit of U.S. Provisional Patent Application No. 63/455,400, filed Mar. 29, 2023. The entire contents of the foregoing applications are incorporated herein by reference.

### FEDERALLY SPONSORED RESEARCH OR DEVELOPMENT

This invention was made with government support under grant CA160433 awarded by the National Institutes of Health. The government has certain rights in the invention.

### TECHNICAL FIELD

The present disclosure relates to the area of biotechnology, and more specifically, to measurement of mammalian telomere lengths.

### BACKGROUND

Epithelial cancers comprise nearly 90% of age-related malignancies, but the biology that drives their development with aging is incompletely understood. Telomere shortening is a well characterized mechanism of cellular aging. Telomeres protect chromosomes from fusion; their length is genetically determined and it predicts the onset of replicative senescence. At critical thresholds, short telomere length signals a double strand DNA damage response and a TP53-dependent checkpoint that provokes apoptosis or cellular senescence, thereby inhibiting tumorigenesis in vivo, and improving cancer-free survival in oncogene—as well as tumor suppressor-mediated animal models of cancer. However, in mice where Tp53 loss was also engineered, short telomeres accelerated carcinogenesis and shifted the spectrum of cancers from sarcomas to epithelial tumors. The tumors that formed had hallmarks of chromosome instability including non-reciprocal translocations. In yeast that lack telomerase, chromosomal rearrangement and increased mutation rates have been observed. Recent cell culture-based models of telomere dysfunction that was induced by telomere deprotection reported kataegis and chromothripsis. These seemingly conflicting results as to whether short telomeres promote or protect against cancer have left open the question of how short telomeres influence human cancer risk and contribute to cancer development in humans with aging.

### SUMMARY

Provided herein are methods of treating pulmonary fibrosis or liver disease or bone marrow failure or primary immunodeficiency in a subject having short telomere syndrome (STS), the method comprising: (a) detecting the presence of STS in the subject; and (b) administering a therapy, wherein the therapy does not suppress T cell immunity or wherein the therapy does not lower immunosuppression.

In some embodiments, the method further comprises (a) evaluating a telomere length of the subject to detect the presence of STS in the subject; and (b) evaluating an immune status of the subject to determine whether the subject has T cell immunodeficiency. In some embodiments, the method further comprises detecting a gene mutation in the subject. In some embodiments, the gene mutation comprises a TERT mutation, TR mutation, RTEL1 mutation, DKC1 mutation, TINF2 mutation, NAF1 mutation. PARN mutation, ZCCHC8 mutation, NHP2 mutation, NOP10 mutation, ACD mutation, STN1 mutation, CTC1 mutation, or TCAB1 mutation. In some embodiments, the gene mutation comprises a loss of function mutation or a gain of function mutation. In some embodiments, the gene mutation is selected from the group consisting of an insertion, a deletion, a substitution, a deletion-insertion, a duplication, an inversion, a frameshift, a repeat expansion, a translocation, and combinations thereof.

In some embodiments, the therapy comprises bone marrow transplant, wherein the bone marrow transplant reverses the T cell immunodeficiency.

Also provided herein are methods of identifying a subject as having an increased risk of a squamous cell cancer (SCC), the method comprising: (a) evaluating telomere length of the subject to determine whether the subject has short telomere syndrome (STS); and (b) stratifying the subject based on the subject having STS, thereby identifying the subject as having an increased risk of the SCC. In some embodiments, the subject is further identified as having a lower risk of lung adenocarcinoma, colorectal cancer, pancreatic cancer, thyroid cancer, melanoma, bladder cancer, kidney cancer, non-Hodgkin lymphoma, or uterine cancer.

In some embodiments, the method further comprises detecting a gene mutation in the subject, thereby identifying the subject as having an increased risk of the SCC. In some embodiments, the gene mutation comprises a TERT mutation, TR mutation, RTEL1 mutation, DKC1 mutation, TINF2 mutation. NAF1 mutation. PARN mutation, ZCCHC8 mutation. NHP2 mutation, NOP10 mutation, ACD mutation, STN1 mutation, CTC1 mutation, or TCAB1 mutation. In some embodiments, the gene mutation comprises a loss of function mutation or a gain of function mutation. In some embodiments, the gene mutation is selected from the group consisting of an insertion, a deletion, a substitution, a deletion-insertion, a duplication, an inversion, a frameshift, a repeat expansion, a translocation, and combinations thereof.

In some embodiments, the squamous cell cancer is a head and neck cancer, anal cancer, or skin cancer.

Also provided herein are methods of treating a squamous cell cancer, the method comprising: (a) evaluating a telomere length of the subject to determine whether the subject has short telomere syndrome (STS); (b) evaluating an immune status of the subject to determine whether the subject has T cell immunodeficiency; (c) identifying the subject as having the squamous cell cancer; and (d) administering an anti-cancer treatment to the subject.

In some embodiments, the method further comprises detecting a gene mutation in the subject. In some embodiments, the gene mutation comprises a TERT mutation, TR mutation, RTEL1 mutation, DKC1 mutation, TINF2 mutation, NAF1 mutation, PARN mutation, ZCCHC8 mutation, NHP2 mutation. NOP10 mutation, ACD mutation, STN1 mutation, CTC1 mutation, or TCAB1 mutation. In some embodiments, the gene mutation comprises a loss of function mutation or a gain of function mutation. In some embodiments, the gene mutation is selected from the group consisting of an insertion, a deletion, a substitution, a deletion-insertion, a duplication, an inversion, a frameshift, a repeat expansion, a translocation, and combinations thereof.

In some embodiments, the squamous cell cancer is a head and neck cancer, esophageal, anal cancer, or skin cancer. In some embodiments, the anti-cancer treatment comprises ionizing radiation, a chemotherapeutic agent, a therapeutic antibody, a checkpoint inhibitor, or any combination thereof.

Also provided herein are methods of identifying a subject as having T-cell immunodeficiency, the method comprising: (a) evaluating telomere length of the subject to determine whether the subject has short telomere syndrome (STS); and (b) stratifying the subject based on the subject having STS, thereby identifying the subject as having T-cell immunodeficiency.

In some embodiments, the method further comprises detecting a gene mutation in the subject. In some embodiments, the gene mutation comprises a TERT mutation, TR mutation, RTEL1 mutation, DKC1 mutation, TINF2 mutation, NAF1 mutation, PARN mutation, ZCCHC8 mutation, NHP2 mutation. NOP10 mutation, ACD mutation, STN1 mutation, CTC1 mutation, or TCAB1 mutation. In some embodiments, the gene mutation comprises a loss of function mutation or a gain of function mutation. In some embodiments, the gene mutation is selected from the group consisting of an insertion, a deletion, a substitution, a deletion-insertion, a duplication, an inversion, a frameshift, a repeat expansion, a translocation, and combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1H show cancer risk in the short telomere syndromes. FIG. 1A shows telogram of individuals in the Johns Hopkins Telomere Syndrome Registry with each individual's germline mutant gene (key) relative to the age-adjusted, clinically validated nomogram. Data are shown for 217 of 226 individuals for whom lymphocyte telomere length data were available. FIG. 1B shows cumulative incidence of hematologic malignancies and solid tumors by age accounting for competing risk of death. FIG. 1C shows distribution of 40 cancers identified in 35 individuals. FIG. 1D shows observed cases of cancer in the Short Telomere Syndrome study participants relative to expected as derived from the Surveillance, Epidemiology, and End Results (SEER) Database and corrected for age and sex. The observed/expected (O/E) ratio is shown to the right. Lung cancer squamous cell cancer (SCC) was excluded from the all SCC comparison. FIG. 1E shows representative hematoxylin and eosin images (100×) of solid tumors: (a) primary SCC of tongue, (b) tongue SCC with involved local lymph node, (c) lip invasive SCC with perineural invasion, (d) gingival SCC, (e) larynx SCC, and (f) anal squamous carcinoma in situ of gluteal cleft which later progressed to anal SCC. FIG. 1F shows clinical characteristics of 14 individuals with 16 cancers (2 individuals with two malignancies are noted by brackets). HPV status was by determined by whole genome sequencing, and for anal cancers, was presumed positive given known association. *Denotes a donor-derived lung adenocarcinoma that developed after transplantation for pulmonary fibrosis. FIG. 1G shows cumulative incidence of cancers in male DKC1 mutation carriers relative to males and females with non-DKC1 mutations. FIG. 1H shows prevalence of all solid tumors in males (M) and females (F) in the Johns Hopkins study relative to age at the time of study enrollment and telomere length.

FIGS. 2A-2L show somatic landscape of solid tumors arising in the short telomere syndromes. FIG. 2A shows TP53 alterations and telomere maintenance mechanisms analyzed by whole genome sequencing. Bottom row shows relative telomere length differences for tumors available for measurement compared to normal as summarized in panel D. FIG. 2B shows somatic alteration frequencies relative to 44 Head and Neck Squamous Cell Carcinoma (HNSCC) from the International Cancer Genome Consortium (ICGC) (n=44 total, median age 57, 52% oral cavity SCC). FIG. 2C shows tumor mutation burden (TMB), calculated as the number of non-synonymous single nucleotide variants and indels per Mb of coding region, is plotted relative to HNSCC ICGC data. FIG. 2D shows relative change in telomere length as measured by quantitative fluorescence in situ hybridization (Q-FISH) of tumors and surrounding normal tissue. FIGS. 2E-2G show representative fluorescence microscopy images (100×) with tumor (T) and normal (N) delineated respectively. Telomere fluorescent probe is in red and DAPI in blue. FIG. 2H shows number of structural variants (SVs) in short telomere syndrome SCC relative to HNSCC ICGC tumors with mean and 25th and 75th percentile lines marked. *Denotes the SCC of skin and esophagus. FIGS. 21-2J show two representative circos plots showing the spectrum of genomic alterations in specified short telomere solid tumors with listed TP53 status above. FIG. 2K shows proportion of SV types for each tumor. FIG. 2L shows shortest distance from each translocation breakpoint to the start of the nearest telomere region plotted. Mean with standard error of the mean are shown.

FIGS. 3A-3G show short telomere patients with solid tumors have T cell immunodeficiency. FIG. 3A show T cell immunodeficiency, both primary and secondary (in some cases both), in 14 individuals with germline mutations in telomere maintenance genes who developed solid tumors. FIG. 3B shows swimmer plot showing interval in years from initiation of immunosuppression to diagnosis of solid tumor in 6 individuals: 4 who underwent solid organ transplant, and 2 who were treated with immunosuppression for presumed autoimmune disease. FIG. 3C-3D shows sagittal and axial computed tomography images (respectively) for a TERT mutation carrier who was diagnosed with a laryngeal squamous cancer (arrows) after lung transplantation for idiopathic pulmonary fibrosis. Images show rightward tracheal displacement due to mass effect. FIG. 3E shows sagittal magnetic resonance image of gingival squamous cell cancer (arrow) diagnosed after initiation of mycophenolate for hypersensitivity pneumonitis prior to short telomere syndrome diagnosis. FIG. 3F shows peripheral blood absolute CD4 and CD8 T cell counts in wildtype. TR−/−G1 and TR−/−G5 mice. FIG. 3G shows CD4:CD8 ratio of mice in FIG. 3F. Mice were on average 16 weeks (range 5-23): Wildtype [7 males (M)/7 females (F)], TR−/−GT (5M/2F), and TR−/−G5 (3M/5F). For FIGS. 3F and 3G, mean and standard error of the mean are plotted. *P<0.05 and ***P<0.001 (Student's t-test, two-sided).

FIG. 4A-4E show impaired immune surveillance of tumors in late generation telomerase null mice is associated with T cell exhaustion. FIG. 4A shows tumor volume relative in mice injected with OvaB6 tumorigenic cells subcutaneously. Each line represents tumor size measured over the time course in one flank. Mice studied were on average 16 weeks old (range 6-24): Wildtype [24 flanks, 12 mice, 6 male (M)/6 female (F)], TR−/−G1 (14 flanks, 7 mice, 4M/3F), TR−/−G5 (64 flanks, 32 mice, 16M/16F). FIG. 4B shows CD3, CD4 and CD8 immunohistochemistry in tumors harvested at each time point and for each genotype shown in FIG. 4A. At day 7, tumors showed comparable burdens of tumor infiltrating T lymphocytes (TILs) but TILs subsequently vanished by day 30 in TR−/−G5 tumors. FIGS. 4C-4E show TIL quantification per tumor area at the timepoints shown in FIG. 4B, with violin plots with median and 25th/75th percentile lines marked. For day 7, wildtype (n=6-7 flanks, 4 mice, 2M/2F), TR−/−GT (6-7 flanks, 7 mice, 5M/2F), and TR−/− G5 (7 flanks, 5 mice, 3M/2F) are shown. For day 30, data for TR−/−G5 (6 flanks, 4 mice, 2M/2F) are shown. **P<0.01 and ***P<0.001 (Mann-Whitney test, corrected for multiple comparisons).

FIGS. 5A-5D show telomere length distribution by quantitative in situ hybridization (Q-FISH) in tumors and adjacent normal tissue (supplementary to FIGS. 2A-2L). FIGS. 5A-5B show distribution of two tumors where the telomere length in the tumor had a very low signal, below detection in most cells relative to normal. These individuals had advanced stage disease. FIGS. 5C-5D show two tumors which have a longer telomere signal relative to normal. Immunofluorescent inset panels show no evidence of ultrabright telomere foci characteristic of the alternative lengthening of telomeres (ALT) mechanism. In parallel, immunohistochemistry insets of the same respective tumors show retained ATRX staining in both tumors suggesting the longer telomeres were maintained via an ATRX-independent mechanism.

FIG. 6 shows COSMIC single base substitution (SBS) mutational signatures in 8 solid tumors derived from individuals with short telomere syndromes (supplementary to FIGS. 2A-2L). Each plot shows the distribution of mutations across the six potential types of substitutions generated by Mutalisk. Mutational signature analysis resulted in cosine score values above 0.90. Signatures were further analyzed using SigProfilerSingleSample and the concordant, predominant signature is annotated for each tumor in addition to any secondary or weaker signatures, if present. Weaker signatures were defined as less than 10% of the subtype detected and secondary as greater than or equal to 10% but less than the predominant signature.

FIG. 7 shows rainfall plot analysis of kataegis focal hypermutability pattern in 8 solid tumors analyzed by high pass whole genome sequencing (supplementary to FIGS. 2A-2L). Each panel represents one tumor's analysis. Each dot represents a somatic single nucleotide variant, and dots are ordered on the horizontal axis according to genomic position. The vertical axis indicates the genomic distance between consecutive variants. None of the plots display a clustering of mutations over a 2 kb-sized DNA region as is characteristic of kataegis. All the plots were generated using a Bambino-based variant calling pipeline, except the squamous cell cancer of the skin plot which was generated using Mutect2 due to high background noise.

FIG. 8 shows circos plots of genomic alterations in short telomere solid cancers (supplementary to FIGS. 2A-2L). Genomic circos plots displaying somatic structural variants as well as greater than 3 Mb copy number variants detected using Illumina whole genome sequencing. Each circos plot is labelled by the cancer type and TP53 status above. Circos plots for 6 tumors are shown with remaining 2 tumors shown in FIGS. 21 and 2J.

FIGS. 9A-9B show tumor size by sex and ovalbumin immunohistochemistry of relapsed tumors (supplementary to FIGS. 4A-4E). FIG. 9A shows maximum tumor volume in the first 10 days with each datapoint representing a single flank in each of the male and female mice studied. Mean and standard error bars are shown. P-value reflects two-sided Mann-Whitney test calculation. FIG. 9B shows immunohistochemistry of tumors harvested after day 30 post-subcutaneous implantation that were harvested from short telomere mice (mTR−/−G5). Three representative images show retained cytoplasmic staining of the ovalbumin antigen (all 8 tumors showed a similar pattern). Tumors were harvested at a mean of 32 days post-implantation (range 24-47).

### DETAILED DESCRIPTION

Short telomere syndromes (STS) are archetypal premature aging syndromes in that they capture a defect acquired universally with aging. They are the most prevalent of premature aging syndromes because of their high incidence among adults with pulmonary fibrosis. Loss-of-function mutations in the telomerase reverse transcriptase, TERT, is the most common cause of STS followed by mutations in the telomerase RNA component, TR. Myelodysplastic syndrome and acute myeloid leukemia comprise the most common malignancies in STS, wherein these cancers arise under selective pressures of telomere-mediated stem cell failure. In this context, somatic reversion mutations that offset the germline loss-of-function mutation in telomerase protect against myeloid malignant evolution. Whether solid tumors that arise in the STS recapitulate the adenocarcinoma-predominant malignancy spectrum associated with aging has not been studied. Moreover, fundamentally, how these tumors overcome the inherited telomere maintenance defect to sustain their evolution is not known.

Provided herein are methods that analyze the somatic landscape of cancers derived from humans with short telomere syndromes (STS), wherein patients with STS are predisposed to developing cancer that is believed to stem from chromosome instability in neoplastic cells. In some embodiments, patients with STS are predisposed to squamous cell carcinoma of the head and neck, anus or skin. In some embodiments, the cancers acquire TERT promoter mutations. In some embodiments, the STS patients' predisposition to solid cancers can be due to T cell immunodeficiency.

Provided herein are methods of identifying a subject as having T-cell immunodeficiency and/or an increased risk of a squamous cell cancer (SCC), the method including (a) evaluating telomere length of the subject to determine whether the subject has short telomere syndrome (STS); and (b) stratifying the subject based on the subject having STS, thereby identifying the subject as having T-cell immunodeficiency and/or an increased risk of a squamous cell cancer.

Also provided herein are methods of treating a squamous cell cancer, the method including:

(a) evaluating a telomere length of the subject to determine whether the subject has short telomere syndrome (STS); (b) evaluating an immune status of the subject to determine whether the subject has T cell immunodeficiency; (c) identifying the subject as having the squamous cell cancer; and (d) administering an anti-cancer treatment to the subject.

Also provided herein are methods of treating pulmonary fibrosis or liver disease or bone marrow failure or primary immunodeficiency in a subject having short telomere syndrome (STS), the method including: (a) detecting the presence of STS in the subject; and (b) administering a therapy, wherein the therapy does not suppress T cell immunity or wherein the therapy does not lower immunosuppression.

Various non-limiting aspects of these methods are described herein, and can be used in any combination without limitation.

It must be noted that, as used in the specification and the appended claims, the singular forms “a,” “an” and “the” include plural referents unless the context clearly dictates otherwise.

As used herein, the term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that are within 25%, 20%, 19%, 18%, 17%, 16%, 15%, 14%, 13%, 12%, 11%, 10%, 9%, 8%, 7%. 6%, 5%, 4%, 3%, 2%, 1%, or less of the referred value.

As used herein, the term “biological sample” refers to a sample obtained from a subject for analysis using any of a variety of techniques including, but not limited to, biopsy, surgery, and laser capture microscopy (LCM), and generally includes cells and/or other biological material from the subject. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. In some embodiments, biological samples can include one or more diseased cells. A diseased cell can have altered metabolic properties, gene expression, protein expression, and/or morphologic features. Examples of diseases include inflammatory disorders, metabolic disorders, nervous system disorders, and cancer.

The biological sample can include any number of macromolecules, for example, cellular macromolecules and organelles (e.g., mitochondria and nuclei). In some embodiments, the biological sample can be a nucleic acid sample and/or protein sample. In some embodiments, the biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As used herein, biological samples can include but are not limited to plasma, serum, blood, tissue, tumor sample, stool, sputum, saliva, urine, sweat, tears, ascites, bronchoaveolar lavage, semen, archeologic specimens and forensic samples. In some embodiments, the biological sample is a solid biological sample (e.g., a tumor sample). In some embodiments, the biological sample is a liquid biological sample. Liquid biological samples can include, but are not limited to plasma, serum, blood, sputum, saliva, urine, sweat, tears, ascites, bronchoaveolar lavage, and semen. In some embodiments, the liquid biological sample is cell free or substantially cell free. In some embodiments, the biological sample is a plasma or serum sample. In some embodiments, the liquid biological sample is a whole blood sample. In some embodiments, the liquid biological sample comprises peripheral mononuclear blood cells. In some embodiments, the biological sample is a cerebrospinal fluid (CSF) sample.

As used herein, the terms “cancer”. “malignancy”, “neoplasm”, “tumor”, and “carcinoma”, refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders: sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

As used herein, the term “subject” is intended to refer to any subject. In some embodiments, the subject is cat, a dog, a goat, a human, a non-human primate, a rodent (e.g., a mouse or a rat), a pig, or a sheep. In some embodiments, a subject is suffering from a relevant disease, disorder or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Telomeres and Telomere Length

As used herein, a “telomere” is a region of repetitive nucleotide sequences associated with specialized proteins at the ends of linear chromosomes. Telomeres are a genetic feature most commonly found in eukaryotes, and are known to protect the terminal regions of chromosomal DNA from progressive degradation and ensure the integrity of linear chromosomes by preventing DNA repair systems from mistaking the very ends of the DNA strand for a double strand break. Telomeres are also critical for maintaining genomic integrity and may be factors for age-related diseases (e.g., bone marrow failure, pulmonary fibrosis, immunodeficiency and other related Telomere Syndromes). Laboratory studies and clinical observations have shown that telomere shortening and dysfunction is commonly acquired due process of cellular aging and tumor development, wherein observational studies have found shortened telomeres in many types of experimental cancers (e.g., squamous cell carcinomas of the skin and upper aerodigestive tract, myelodysplasia, and acute myeloid leukemia). Despite having short telomeres, cancer cells typically have activated telomerase that will maintain telomeres.

Most vertebrate telomeric DNA consists of long (TTAGGG)n repeats of variable length, often around 3-20 kb. A subtelomere is a segment of DNA between telomeric caps and coding regions of DNA, wherein in vertebrates, each chromosome has two subtelomeres immediately adjacent to the long (TTAGGG)n repeats at each chromosome end. In some embodiments, subtelomeres are considered to be the most distal (farthest from the centromere) region of unique DNA on a chromosome, and they are unusually dynamic and variable mosaics of highly repeated blocks of sequence.

Telomere length varies greatly between species, from approximately 300 base pairs in yeast to many kilobases in humans, and usually is composed of arrays of guanine-rich, six- to eight-base-pair-long repeats. Eukaryotic telomeres normally terminate with 3′ single-stranded-DNA overhang, which is essential for telomere maintenance and capping. Telomere length also varies within the human population.

### Methods of Evaluating Telomere Length

In some embodiments, the method further includes analyzing the biological sample to measure telomere length in a cell from the biological sample. In some embodiments, quantification of telomere length can comprise Flow-FISH. In some embodiments, quantification of telomere length can comprise whole genome sequencing. In some embodiment, quantification of telomere length can comprise whole genome sequencing of DNA using various methods (e.g., short and long read). In some embodiments, quantification of telomere length can comprise terminal restriction fragmentation, quantitative-PCR, or use of fluorescent probes to quantify chromosome-specific telomere lengths. In some embodiments, quantification of telomere length can comprise terminal restriction fragmentation (TRF). In some embodiments, quantification of telomere length can comprise polymerase chain reaction (PCR)-based techniques (e.g., qPCR, MMqPCR, and aTL qPCR). In some embodiments, quantification of telomere length can comprise single telomere length analysis (STELA). In some embodiments, quantification of telomere length can comprise quantitative fluorescence in situ hybridization (Q-FISH). In some embodiments, quantification of telomere length can comprise metaphase chromosome Q-FISH. In some embodiments, quantification of telomere length can comprise interphase Q-FISH. In some embodiments, quantification of telomere length can comprise a primed in situ (PRINS) approach. In some embodiments, quantification of telomere length can comprise a hybridization protection assay (HPA). In some embodiments, quantification of telomere length can comprise single-strand 3′-overhang measurement. In some embodiments, the telomere length is measured by performing qPCR, Flow cytometry-FISH (flowFISH) in leukocyte subsets, or both.

As used herein, “short telomere syndromes (STS)” refer to accelerated aging syndromes often caused by inheritable gene mutations resulting in decreased telomere lengths. In some embodiments, as a consequence, organs systems with increased cell turnover and/or secondary damage with aging (e.g., skin, bone marrow, lungs, and the gastrointestinal tract) are commonly affected. Due to diverse clinical presentations, STS pose a diagnostic challenge, with bone marrow failure and idiopathic pulmonary fibrosis being frequent manifestations. In some embodiments, STS can occur in association with gene mutations involving DKC1 (dyskerin), TERT (telomerase reverse transcriptase) and TERC (telomerase RNA component) amongst others. In some embodiments lower measured levels of these genes may signify a STS even in the absence of a readily identifiable gene mutation.

In some embodiments, STS can include bone marrow failure syndromes (e.g., aplastic anemia), dyskeratosis congenita, pulmonary fibrosis, emphysema, cryptogenic liver cirrhosis, lacrimal ductal, esophageal and urethral stenosis, premature graying of hair, avascular necrosis of hips and shoulder, periodontal disease and an increased predisposition towards epithelial and hematological malignancies. In some embodiments, STS can also include idiopathic pulmonary fibrosis (IPF), bronchiolitis obliterans with organizing pneumonia, chronic hypersensitivity pneumonitis, interstitial pneumonitis and emphysema. In some embodiments, STS can include hepatic manifestations such as hepatic parenchymal inflammation, hepatic fibrosis, cryptogenic cirrhosis of the liver, nodular regenerative hyperplasia of the liver (NRH), and portal hypertension. In some embodiments, STS can include gastrointestinal manifestations such as esophageal stenosis, B-cell immunodeficiency with enterocolitis, and a celiac like enteropathy (telomere-mediated stem cell failure). In some embodiments, STS can include apoptosis or early senescence of B, NK and T lymphocytes and a spectrum of immune defects (e.g., immunodeficiency).

Methods of Identifying a Subject as Having T-Cell Immunodeficiency and/or an Increased Risk of a Squamous Cell Cancer (SCC)

Provided herein are methods of identifying a subject as having T-cell immunodeficiency, the method comprising: (a) evaluating telomere length of the subject to determine whether the subject has short telomere syndrome (STS); and (b) stratifying the subject based on the subject having STS, thereby identifying the subject as having T-cell immunodeficiency. Also provided herein are methods of identifying a subject as having an increased risk of a squamous cell cancer (SCC), the method comprising: (a) evaluating telomere length of the subject to determine whether the subject has short telomere syndrome (STS); and (b) stratifying the subject based on the subject having STS, thereby identifying the subject as having an increased risk of the SCC.

As used herein, the term “immunodeficiency” refers to a deficiency that results from a failure or absence of elements of the immune system (e.g., lymphocytes, phagocytes) and the complement system. In some embodiments, immunodeficiency can include B-cell deficiencies, selective immunoglobulin IgA deficiencies, T-cell immunodeficiencies, or T-cell and B-cell deficiencies. As used herein. “T-cell immunodeficiencies” are a group of disorders that affect the development, activation, survival and diversity of T cells. T-cell immunodeficiencies can affect T cells in isolation or in conjunction with other cell types, including B cells, NK cells, and components of the innate immune system.

As used herein, the term “stratify” refers to assigning a treatment regimen. In some embodiments, a subject can be stratified and placed in a therapy category, wherein a treatment regimen in the therapy category is assigned to the subject. In some embodiments, stratification of a subject can be used in prospective or retrospective clinical studies. In some embodiments, stratification of a subject can be used to assign a prognosis or a prediction regarding survival or chemotherapy or radiotherapy sensitivity. In some embodiments, stratification typically assigns a subject to a group based on a shared mutation pattern or other observed characteristic or set of characteristics. In some embodiments, stratification of a subject can be based on the subject having short telomere syndrome. In some embodiments, stratification of the subject as having STS can result in identifying the subject as having T-cell immunodeficiency. In some embodiments, stratification of the subject as having STS can result in identifying the subject as having an increased risk of a squamous cell cancer. In some embodiments, the squamous cell cancer is a head and neck cancer, esophageal, anal cancer, or skin cancer. In some embodiments, the subject can be further identified as having a lower risk of lung adenocarcinoma, colorectal cancer, pancreatic cancer, thyroid cancer, melanoma, bladder cancer, kidney/renal pelvis cancer, non-Hodgkin lymphoma, or uterine cancer.

In some embodiments, the method further includes detecting a gene mutation in the subject, thereby identifying the subject as having an increased risk of the SCC. In some embodiments, the gene mutation comprises a TERT mutation, TR mutation, RTEL1 mutation, DKC1 mutation, TINF2 mutation, NAF1 mutation, PARN mutation, ZCCHC8 mutation, NHP2 mutation, NOP10 mutation, ACD mutation, STN1 mutation, CTC1 mutation, or TCAB1 mutation. In some embodiments, the gene mutation comprises a loss of function mutation or a gain of function mutation. In some embodiments, the gene mutation is selected from the group consisting of an insertion, a deletion, a substitution, a deletion-insertion, a duplication, an inversion, a frameshift, a repeat expansion, a translocation, and combinations thereof.

### Therapeutic Applications

Provided herein are method of treating a squamous cell cancer, the method comprising. (a) evaluating a telomere length of the subject to determine whether the subject has short telomere syndrome (STS); (b) evaluating an immune status of the subject to determine whether the subject has T cell immunodeficiency; (c) identifying the subject as having the squamous cell cancer; and (d) administering an anti-cancer treatment to the subject.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e, g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

In some embodiments, the anti-cancer treatment can include ionizing radiation, a chemotherapeutic agent, a therapeutic antibody, a checkpoint inhibitor, or any combination thereof. In some embodiments, the anti-cancer treatment can be administered to a subject having a cancer as a combination therapy with one or more additional cancer treatments. An anti-cancer treatment can include any appropriate cancer treatments. For example, an anti-cancer treatment can include surgery. For example, an anti-cancer treatment can include radiation therapy. For example, an anti-cancer treatment can include administration of one or more therapeutic agents (e.g., one or more anti-cancer agents). In some embodiments, an anti-cancer agent can be an immunotherapy (e.g., a checkpoint inhibitor). In some embodiments, the subject has previously been administered one or more additional anti-cancer therapies selected from the group consisting of ionizing radiation, a chemotherapeutic agent, a therapeutic antibody, or a checkpoint inhibitor. In some embodiments, the subject will be administered one or more additional anticancer therapies selected from the group consisting of ionizing radiation, a chemotherapeutic agent, a therapeutic antibody, or a checkpoint inhibitor.

Also provided herein are methods of treating pulmonary fibrosis or liver disease or bone marrow failure or primary immunodeficiency in a subject having short telomere syndrome (STS), the method comprising: (a) detecting the presence of STS in the subject; and (b) administering a therapy, wherein the therapy does not suppress T cell immunity or wherein the therapy does not lower immunosuppression. In some embodiments, the method can further comprise (a) evaluating a telomere length of the subject to detect the presence of STS in the subject; and (b) evaluating an immune status of the subject to determine whether the subject has T cell immunodeficiency.

In some embodiments, the therapy that does not suppress T cell immunity nor lower immunosuppression can include a therapy that reverses immunodeficiency in the subject. In some embodiments, the therapy can include hematopoietic stem cell transplantation or hematopoietic stem cell gene therapy. In some embodiments, the therapy can include bone marrow transplant, wherein the bone marrow transplant reverses the T cell immunodeficiency in the subject. In some embodiments, the therapy can include administration of medications that decrease a risk of infections (e.g., cytomegalovirus or Pneumocystis jirovecii). In some embodiments, the therapy can include increased monitoring of the subject with T cell immunodeficiency.

### EXAMPLES

The disclosure is further described in the following examples, which do not limit the scope of the disclosure described in the claims.

### Example 1—Solid Tumors in the Short Telomere Syndromes have a Distinct Spectrum from Age-Related Malignancies

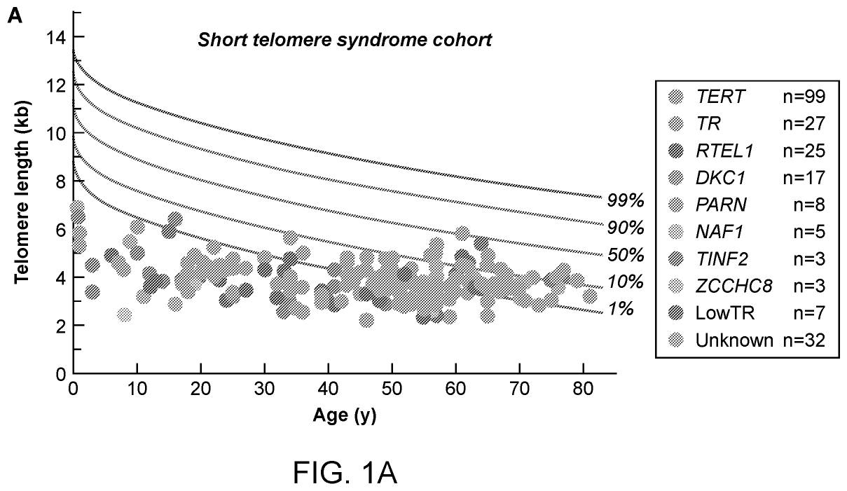

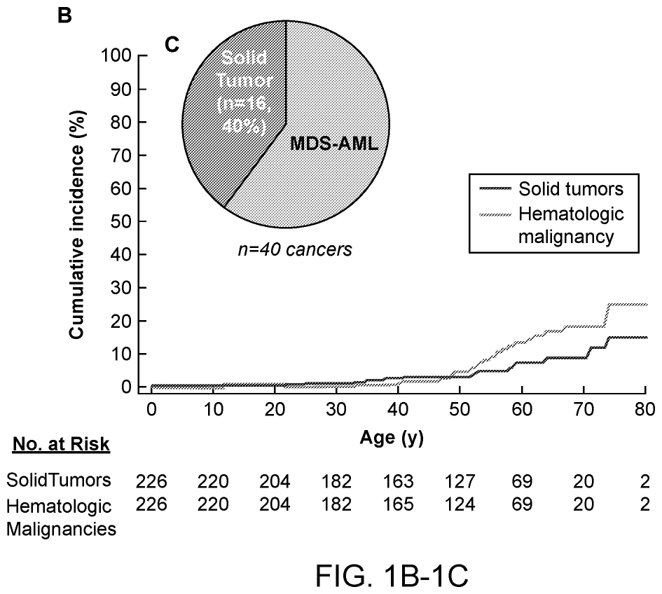

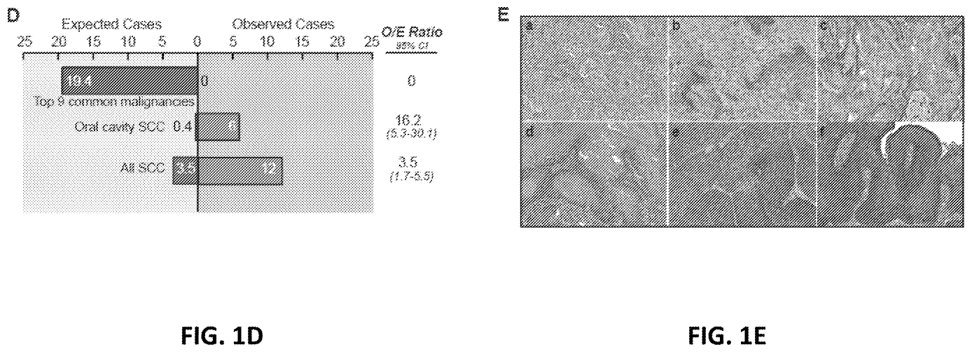

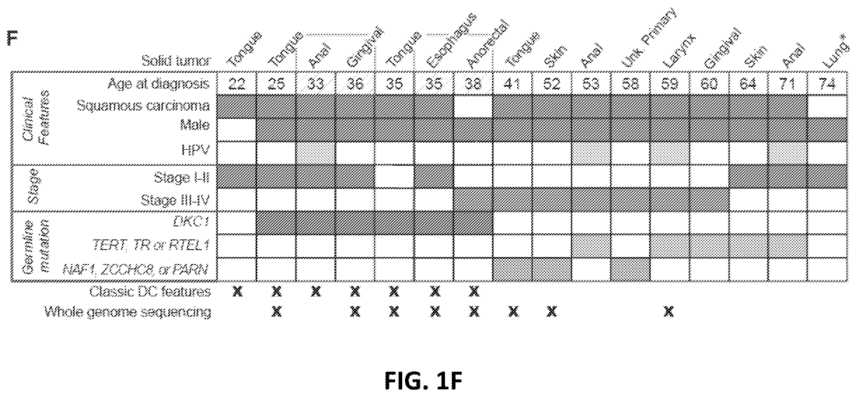

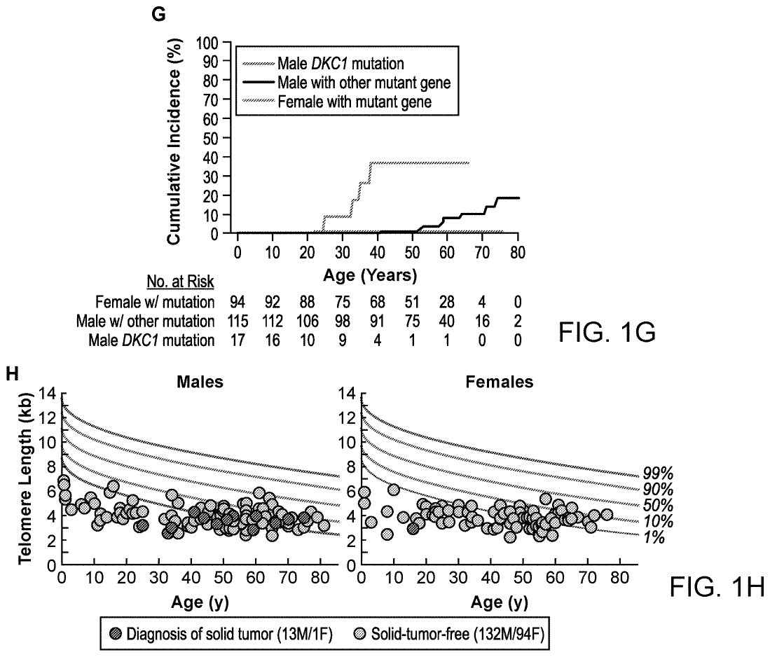

To understand the role of short telomeres in human carcinogenesis, the prevalence of invasive solid tumors was assessed among 226 individuals with STS who were recruited from 2003-2022 (FIG. 1A and clinical characteristics in Table 1). Median age at enrollment was 50 (range 0.6-81 years, 10,890 lifetime-years) and 58% were male. TERT loss-of-function mutations were most common (44%), followed by TR, RTEL1 and DKC1 (FIG. 1A and Table 1).

TABLE 1

| | Whole | | Females | | Males | | |
|---|---|---|---|---|---|---|---|
| | Cohort | | N = 94 | | N = 132 | | |
| | N = 226 | | (41.6%) | | (58.4%) | | P** |
| Age at Enrollment - mean (SD) | 45.44 | (19.47) | 44.1 | (18.17) | 46.4 | (20.37) | ns |
| Age at Enrollment - median (range) | 50 | (0.58, 81) | 49 | (1, 76) | 51 | (0.58, 81) | ns |
| Deceased as of data cutoff | | | | | | | |
| No | 105 | (46.5) | 55 | (58.5) | 50 | (37.9) | 0.008 |
| Unknown | 60 | (26.5) | 20 | (21.3) | 40 | (30.3) | |
| Yes | 61 | (27) | 19 | (20.2) | 42 | (31.8) | |
| Sex | | | | | | | |
| Female | 94 | (41.6) | 94 | (100) | 0 | (0) | <0.001 |
| Male | 132 | (58.4) | 0 | (0) | 132 | (100) | |
| Mutant Gene | | | | | | | |
| DKC1 - | 17/226 | (7.5) | 0/94 | (0) | 17/132 | (12.9) | <0.001 |
| RTEL1 | 25/226 | (11.1) | 11/94 | (11.7) | 14/132 | (10.6) | ns |
| TERT | 99/226 | (43.8) | 49/94 | (52.1) | 50/132 | (37.9) | 0.041 |
| TINF2 | 3/226 | (1.3) | 2/94 | (2.1) | 1/132 | (0.8) | ns |
| TR | 27/226 | (11.9) | 18/94 | (19.1) | 9/132 | (6.8) | 0.006 |
| ZCCHCB | 3/226 | (1.3) | 0/94 | (0) | 3/132 | (2.3) | ns |
| NAF1 | 5/226 | (2.2) | 4/94 | (4.3) | 1/132 | (0.8) | ns |
| PARN | 8/226 | (3.5) | 2/94 | (2.1) | 6/132 | (4.5) | ns |
| Transplant status | | | | | | | |
| Any Transplant | 51/226 | (22.6) | 16/94 | (17) | 35/132 | (26.5) | ns |
| Received BMT | 17/226 | (7.5) | 8/94 | (8.5) | 9/132 | (6.8) | ns |
| Received Solid Organ Transplant | 36/226 | (15.9) | 9/94 | (9.6) | 27/132 | (20.5) | 0.028 |
| Cancer diagnoses* | | | | | | | |
| Any Cancer | 35/226 | (15.5) | 7/94 | (7.4) | 28/132 | (21.2) | 0.005 |
| Solid Tumor | 14/226 | (6.2) | 1/94 | (1.1) | 13/132 | (9.8) | 0.009 |
| MDS or AML | 24/226 | (10.6) | 6/94 | (6.4) | 18/132 | (13.6) | ns |

Participants had very short telomeres relative to age-matched healthy controls with 93% falling below the 10^{th} percentile (n=201) and 64% below the 1^{st} percentile (n=138) (FIG. 1A). Cumulative incidence of solid cancers increased with age, but was overall low [0.09 by age 70, 95% confidence interval (CI) 0.04-0.14, accounting for competing risks of mortality] and lower than the cumulative incidence of hematologic malignancies (0.18 at the same age, 95% CI 0.11-0.26) (FIG. 1B and Table 2). Sixteen invasive solid tumors were diagnosed in 14 individuals and these comprised 40% of all the cancers with the hematologic malignancies being either myelodysplastic syndrome or acute myeloid leukemia (FIG. 1C).

TABLE 2

| | N | N events | Age 50 | Age 60 | Age 70 | HR (95% CI) | P |
|---|---|---|---|---|---|---|---|
| Solid Tumors: Whole Cohort | 226 | 14 | 0.03 (0.01, [symbol] ) | 0.08 (0.03, 0.12) | 0.09 (0.04, 0.14) | | |
| Sex | | | | | | | |
| Females | 94 | 1 | 0.01 (0, 0.03) | 0.01 (0, 0.03) | 0.01 (0, 0.03) | | |
| Males | 132 | 13 | 0.06 (0.01, 0.09) | 0.11 (0.05, [symbol] ) | 0.13 ( [symbol] , 0.21) | 8.01 (1.07, [symbol] ) | 0.0432 |
| Received Solid Organ Transplant | | | | | | | |
| No | 190 | 9 | 0.03 (0, 0.06) | 0.06 (0.01, 0.1) | 0.07 (0.02, 0.12) | | |
| Yes | 36 | 5 | 0.03 (0, 0.08) | 0.14 (0.01, 0.27) | 0.14 (0.01, 0.27) | 2.13 (0.74, 6.1) | 0.1583 |
| DKC1 Mutation-By-Sex | | | | | | | |
| No DKC1 Mutation (Males) | 115 | 9 | 0.01 (0, 0.03) | 0.08 (0.02, 0.15) | 0.1 (0.03, 0.18) | 1.0 (Ref) | 1.0 (Ref) |
| DKC1 Mutation | 17 | 4 | 0.37 ( [symbol] , [symbol] ) | 0.37 ( [symbol] , 0.67) | | 7.02 (2.12, 23.3) | 0.0014 |
| No DKC1 Mutation (Females) | 94 | 1 | 0.01 (0, 0.03) | 0.01 (0, 0.03) | 0.01 (0, 0.03) | 0.17 (0.02, 1.31) | 0.089 |
| MDS/AML: Whole Cohort | 226 | 24 | 0.04 (0.01, 0.08) | [symbol] (0.08, 0.19) | 0.18 (0.11, 0.26) | | |
| Sex | | | | | | | |
| Females | 94 | 6 | 0.02 (0, 0.06) | 0.08 (0, 0.16) | 0.15 (0.03, 0.26) | | |
| Males | 132 | 18 | 0.06 (0.01, 0.11) | 0.16 (0.08, 0.24) | 0.2 (0.11, 0.29) | 1.69 (0.68, 4.23) | [symbol] |
| MDS/AML: DKC1 Mutation-By-Sex | | | | | | | |
| No DKC1 Mutation (Males) | 115 | 16 | 0.04 (0, 0.09) | 0.15 (0.07, 0.24) | 0.2 (0.1, 0.29) | 1.0 (Ref) | 1.0 (Ref) |
| DKC1 Mutation | 17 | 2 | 0.23 (0, 0.59) | 0.23 (0, 0.59) | | 1.97 (0.41, 9.5) | 0.4007 |
| No DKC1 Mutation (Females) | 94 | 6 | 0.02 (0, 0.06) | 0.08 (0, 0.16) | 0.15 (0.03, 0.26) | 0.63 (0.25, 1.58) | 0.3197 |

[symbol]

indicates data missing or illegible when filed

Comparing the adjusted incidence to the National Cancer Institute's Surveillance, Epidemiology, and End Results (SEER) data, there was a lower than expected number of the 9 most common cancers associated with mortality in adults (i.e. lung, colorectal, pancreatic, kidney, bladder, uterine, melanoma, thyroid and non-Hodgkin lymphoma): 19.4 cases expected vs. none documented (FIG. 1D). In contrast to age-related solid tumors, which are predominantly of adenocarcinoma histology, nearly all the solid tumors were derived from the squamous epithelium (88%, 14 of 16, FIG. 1E). The most common sites were the oral cavity and other sites of the head and neck (44%, n=7, FIG. 1F), followed by anal squamous cell cancers (SCC) (19%, n=3) and skin SCC (13%, n=2) (FIG. 1F). The risk of oral cavity SCC and all SCC was increased [Observed/Expected (O/E) ratio 16.2, 95% CI: 5.3-30.1 and O/E ratio 3.5, 95% CI 1.72-5.46, respectively](FIG. 1D). In examining the clinical features, nearly all the patients who developed solid tumors were male (93%, 13 of 14) (FIG. 1F). Males with X-linked DKC1 mutations had the highest risk [Hazard Ratio (HR) 6.93 relative to males with other mutations, 95% CI 1.87-25.68], and females had the lowest risk (HR 0.18, 95% CI 0.02-1.46, FIGS. 1G-1H, Tables 2-3). The data indicated that STS have a distinct solid tumor spectrum from what is seen with aging and it is largely limited to squamous carcinoma.

TABLE 3

| | HR [95% CI] | | P |
|---|---|---|---|
| Model 1 | | | |
| Males v Females | 7.55 | (0.94, 60.43) | 0.0568 |
| Red'd Solid Organ | 1.85 | (0.63. 5.45) | 0.264 |
| Transplant vs. Did Not | | | |
| Model 2 | | | |
| Rec'd Solid Organ | 1.84 | (0.55, 6.16) | 0.3255 |
| Transplant vs. Did Not | | | |
| DKC1 Mutation vs. No | 6.93 | (1.87, 25.68) | 0.0038 |
| DKC1 Mutation (Males) | | | |
| No DKC1 Mutation (Females) vs. | 0.18 | (0.02, 1.46) | 0.1089 |
| No DKC1 Mutation (Males) | | | |

### Example 2—Cancers Acquire Telomere Maintenance Mechanisms and have No Evidence of Telomere Crisis

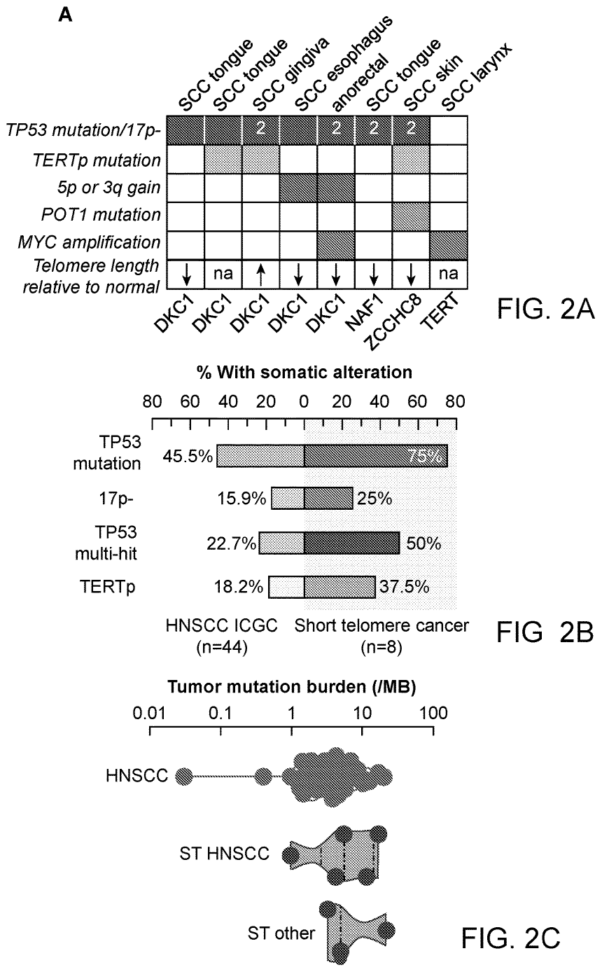

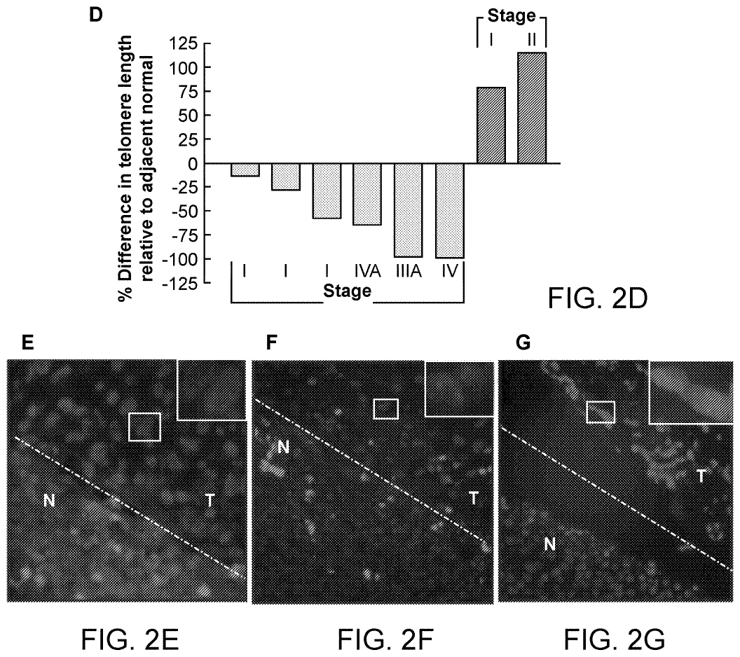

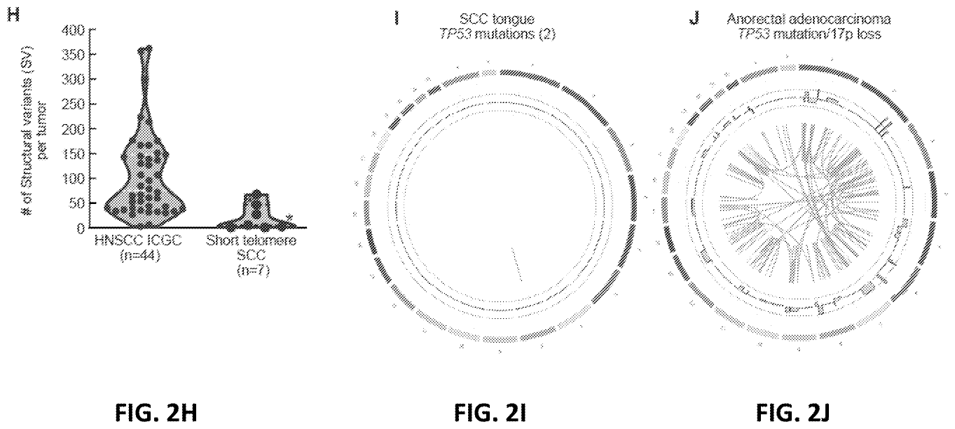

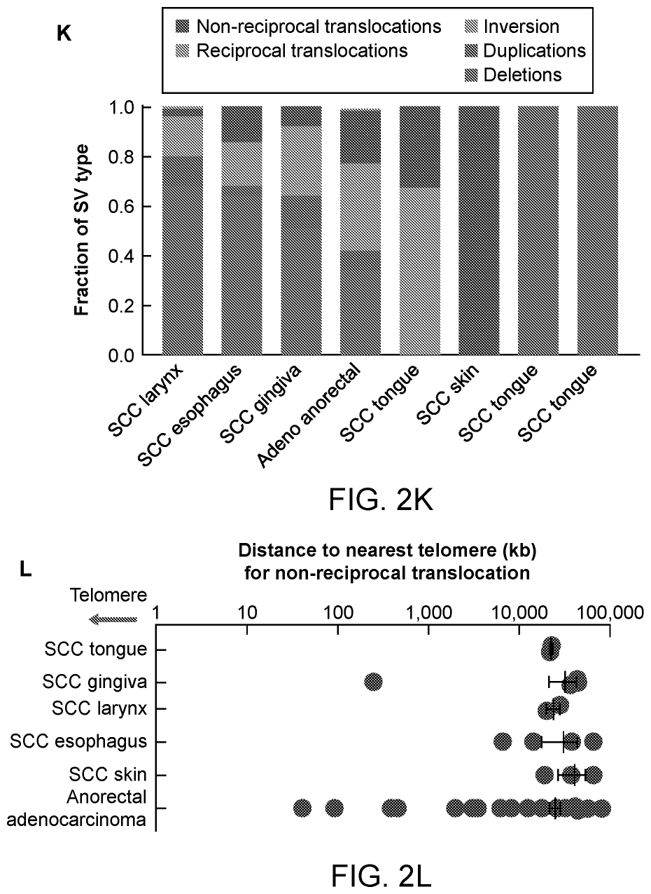

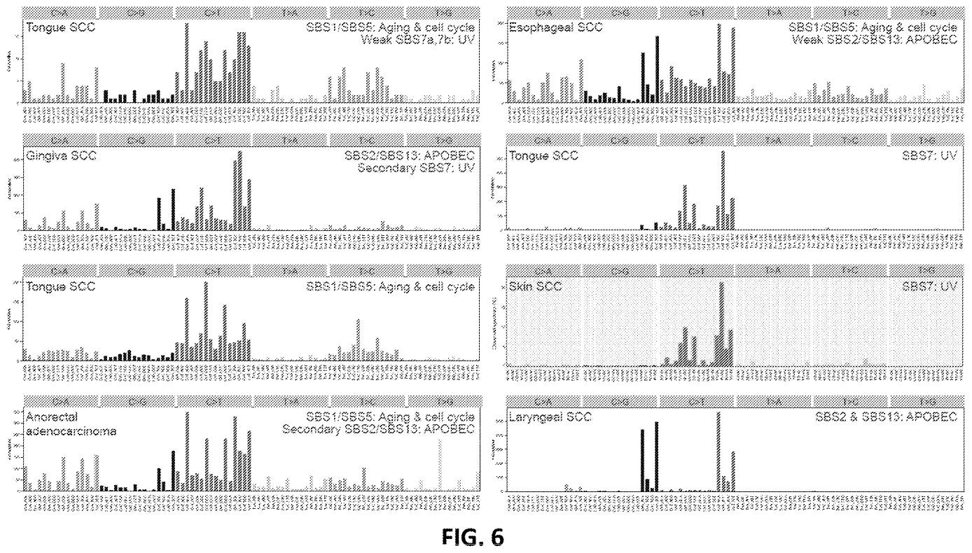

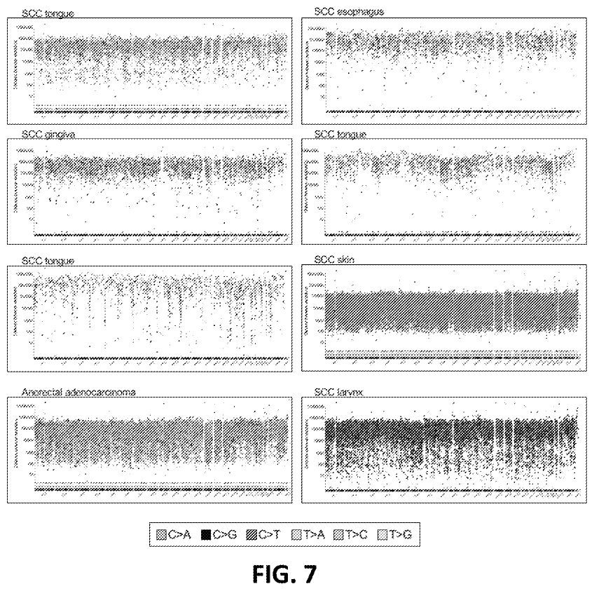

To assess how solid tumors overcome the germline telomere defect, paired tumor-normal high pass whole genome sequencing was performed (mean 72×) on 8 cancers (7 SCC and 1 anorectal adenocarcinoma, FIG. 2A). Nearly all the tumors carried at least one TP53 loss-of-function alteration (88%. 7 of 8), and 50% had two hits (4 of 8. FIG. 2A, Tables 4-5). Most tumors acquired telomere maintenance mechanisms with canonical TERT promoter mutations that upregulate TERT transcription being most common, but there was also amplification of the TERT and TR loci at 5p and 3q, respectively, as well as a POT1 mutation and MYC amplification detected (FIG. 2A). Multiple telomere maintenance mechanisms co-existed in some tumors. No tumor had copy number loss or mutations in alternative lengthening of telomere (ALT)-associated genes, ATRX, DAXX or SMARCAL1. Relative to a comparably sequenced TCGA Head and Neck SCC dataset, the prevalence of TP53 alterations and TERT promoter mutations was higher (FIG. 2B), but the tumor mutation burden was comparable and low (mean 8.7/Mb, range 1.0-22.6, FIG. 2C). The predominant single base substitution signature (SBS) was that of replicative aging (C>T dominant, COSMIC SBS1) which was found in 5 of 8 cancers; it explained 22% of single nucleotide variants (range 13-42%, FIG. 6). Kataegis has been identified in some cell-based telomere deprotection models, but these patterns were not detected and the shortest inter-cluster distance identified was 515 kb, longer than the 2 kb intercluster threshold characteristic of kataegis (FIG. 7). Additionally, STS oral cavity SCC tumors had few deep focal copy number alterations (mean 26/genome, range 8-64), a pattern that was distinct from oral cavity tumors derived from Fanconi anemia patients. The data indicated that solid tumors arising in STS overcome the short telomere checkpoint through loss-of-function of TP53 while simultaneously acquiring telomere maintenance mechanisms.

TABLE 4
Telomere Maintenance Genes**

| | | | | |
|---|---|---|---|---|
| MYC | POT1 | TERC | TERT | TERT |
| | | | | promoter |

Alternative Lengthening of Telomeres (ALT)**

| | | | | | |
|---|---|---|---|---|---|
| ATRX | DAXX | SMARCAL1 | KDM4B | IDH1/2 | H3.3 |

Cell-Cycle Control

| | | | | | |
|---|---|---|---|---|---|
| CCND1 | CDKN2A | FBXW7 | MYC | RB1 | TP53 |

Mitogenesis/RAS Signaling

| | | | | | |
|---|---|---|---|---|---|
| AJUBA | BRAF | DDR2 | EGFR | FGFR1 | FGFR2 |
| FGFR3 | HRAS | KRAS | MAPK1 | NRAS | PIK3CA |
| PTEN | | | | | |

Squamous Cell Differentiation

| | | | | | |
|---|---|---|---|---|---|
| FAT1 | NOTCH1 | NOTCH2 | NOTCH3 | SOX2 | TP63 |

Chromatin/Transcription/Gene Expression Control

| | | | | | |
|---|---|---|---|---|---|
| DDX3 | EP300 | EZH2 | KMT2C | KMT2D | MED1 |
| NSD1 | SYNE1 | | | | |

Cell Survival

| | | | |
|---|---|---|---|
| CASP8 | CUL3 | KEAP1 | NFE2L2 |

### Example 3—Tumor Telomere Length and Chromosome Stability of Tumors

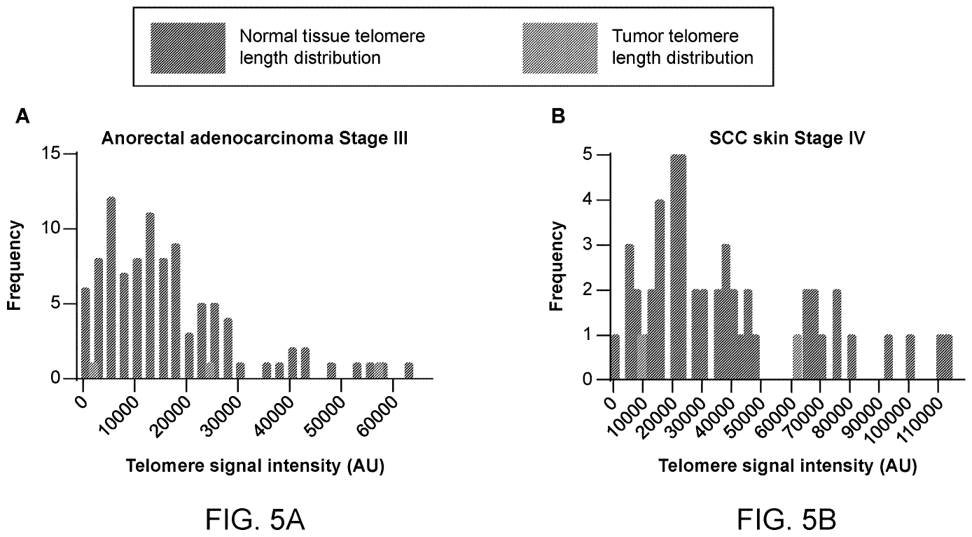

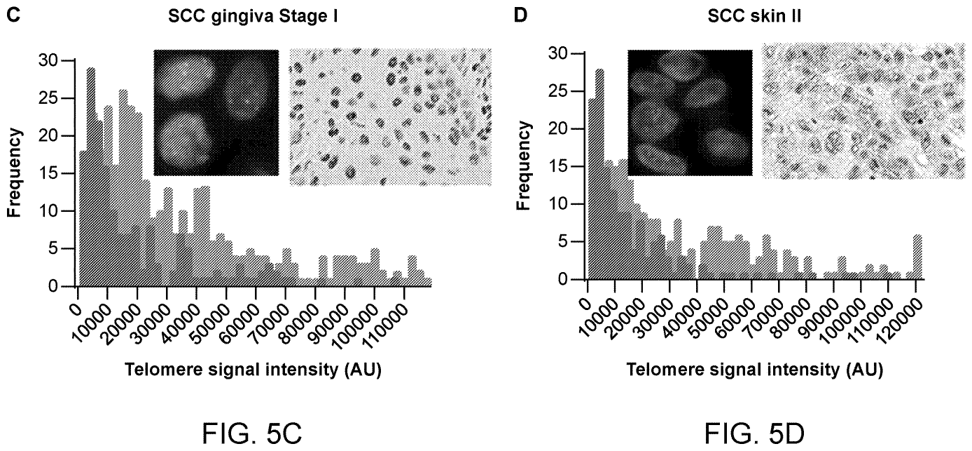

In the context of the inherited telomere maintenance defect, the tumor telomere length was assessed by quantitative fluorescence situ hybridization (FISH). It was found that most cancers had somatic shortening relative to the adjacent normal tissues (6 of 8, 67%) (FIGS. 2D-2F and FIGS. 5A-5B). The extent of shortening was greatest in advanced stage cancers (Stage III-IV relative to Stage I-II) (FIG. 2D). In two cases, the tumor telomere length was longer (FIGS. 2D, 2G and FIGS. 5C-5D), but these cancers did not show ultra-bright telomere foci characteristic of ALT, and retained ATRX staining (FIGS. 5C-5D). Their absence of alterations associated with ALT genes (Table 4) and telomere lengthening without ALT is similar to what is seen in some epithelial cancers with yet-uncharacterized telomere maintenance mechanisms.

TABLE 5

| | | | | | | | | Sequencing Depth (count) | | | | [symbol] | |
|---|---|---|---|---|---|---|---|---|---|---|---|---|---|
| [symbol] | | [symbol] | | | Coding | [symbol] | | Tumor | Tumor | Normal | Normal | [symbol] | COSMIC |
| [symbol] | Gene | coordinates | [symbol] | [symbol] | [symbol] | Change | VA [symbol] | Ref | Att | Ref | Att | Score | [symbol] |
| SCC [symbol] | ATR [symbol] | [symbol] | G | C | [symbol] | [symbol] | [symbol] | [symbol] | 24 | [symbol] | 0 | [symbol] | [symbol] SCC |
| SCC [symbol] | ATR [symbol] | [symbol] | [symbol] | [symbol] | [symbol] | [symbol] | 0.29 | [symbol] | 4 | [symbol] | 0 | [symbol] | Not reported |
| SCC [symbol] | [symbol] | [symbol] | G | A | [symbol] | [symbol] | [symbol] | [symbol] | [symbol] | 118 | 0 | [symbol] | [symbol] |
| SCC [symbol] | [symbol] | [symbol] | G | A | [symbol] | [symbol] | [symbol] | 42 | [symbol] | [symbol] | 0 | [symbol] | Not reported |
| SCC [symbol] | [symbol] | [symbol] | C | [symbol] | [symbol] | [symbol] | [symbol] | [symbol] | 4 | [symbol] | 0 | [symbol] | Not reported |
| SCC [symbol] | [symbol] | [symbol] | C | G | [symbol] | [symbol] | [symbol] | [symbol] | [symbol] | [symbol] | 0 | [symbol] | [symbol] |
| SCC [symbol] | [symbol] | [symbol] | C | G | [symbol] | [symbol] | [symbol] | [symbol] | [symbol] | [symbol] | 0 | [symbol] | [symbol] |
| [symbol] | [symbol] | [symbol] | [symbol] | [symbol] | [symbol] | [symbol] | 0.11 | [symbol] | 8 | 85 | 0 | n/a | Not reported |
| SCC [symbol] | [symbol] | [symbol] | C | G | [symbol] | [symbol] | [symbol] | [symbol] | 12 | [symbol] | 0 | [symbol] | Not reported |
| SCC [symbol] | NOTCH [symbol] | [symbol] | [symbol] | A | [symbol] | [symbol] | [symbol] | [symbol] | [symbol] | [symbol] | 0 | n/a | Not reported |
| SCC [symbol] | [symbol] | [symbol] | G | A | [symbol] | [symbol] | [symbol] | [symbol] | 18 | [symbol] | 0 | [symbol] | [symbol] |
| SCC [symbol] | PTEN | [symbol] | G | A | [symbol] | [symbol] | [symbol] | [symbol] | [symbol] | [symbol] | 0 | [symbol] | [symbol] |
| SCC [symbol] | PTEN | [symbol] | C | [symbol] | [symbol] | [symbol] | 0.40 | [symbol] | 12 | [symbol] | 0 | [symbol] | Not reported |
| SCC [symbol] | [symbol] | [symbol] | A | G | [symbol] | [symbol] | 0.31 | 20 | [symbol] | [symbol] | 0 | [symbol] | Not reported |
| [symbol] | SYN [symbol] | [symbol] | G | T | [symbol] | [symbol] | 0.24 | [symbol] | [symbol] | [symbol] | 0 | [symbol] | [symbol] |
| [symbol] | SYN [symbol] | [symbol] | G | T | [symbol] | [symbol] | [symbol] | 24 | [symbol] | [symbol] | 0 | 22.9 | [symbol] |
| SCC [symbol] | [symbol] | [symbol] | G | A | [symbol] | — | [symbol] | 2 | [symbol] | 85 | 0 | n/a | [symbol] |
| SCC [symbol] | [symbol] | [symbol] | G | A | [symbol] | — | [symbol] | [symbol] | 5 | [symbol] | 0 | n/a | [symbol] |
| SCC [symbol] | [symbol] | [symbol] | G | A | [symbol] | — | [symbol] | [symbol] | [symbol] | [symbol] | 0 | n/a | [symbol] |
| SCC [symbol] | [symbol] | [symbol] | C | T | [symbol] | [symbol] | 0.15 | [symbol] | [symbol] | 111 | 0 | [symbol] | [symbol] |
| [symbol] | [symbol] | [symbol] | G | A | [symbol] | [symbol] | 0.13 | [symbol] | [symbol] | [symbol] | 0 | [symbol] | [symbol] |
| SCC [symbol] | [symbol] | [symbol] | C | T | [symbol] | [symbol] | 0.11 | [symbol] | [symbol] | [symbol] | 0 | [symbol] | [symbol] |
| [symbol] | [symbol] | [symbol] | T | C | [symbol] | [symbol] | [symbol] | [symbol] | [symbol] | [symbol] | 0 | 24 | [symbol] |
| SCC [symbol] | [symbol] | [symbol] | C | T | [symbol] | [symbol] | [symbol] | [symbol] | [symbol] | [symbol] | 0 | [symbol] | [symbol] |
| [symbol] | [symbol] | [symbol] | C | T | [symbol] | [symbol] | [symbol] | [symbol] | [symbol] | [symbol] | 0 | [symbol] | [symbol] |
| [symbol] | [symbol] | [symbol] | G | A | [symbol] | [symbol] | [symbol] | [symbol] | 31 | [symbol] | 0 | 27.7 | [symbol] |

[symbol]

indicates data missing or illegible when filed

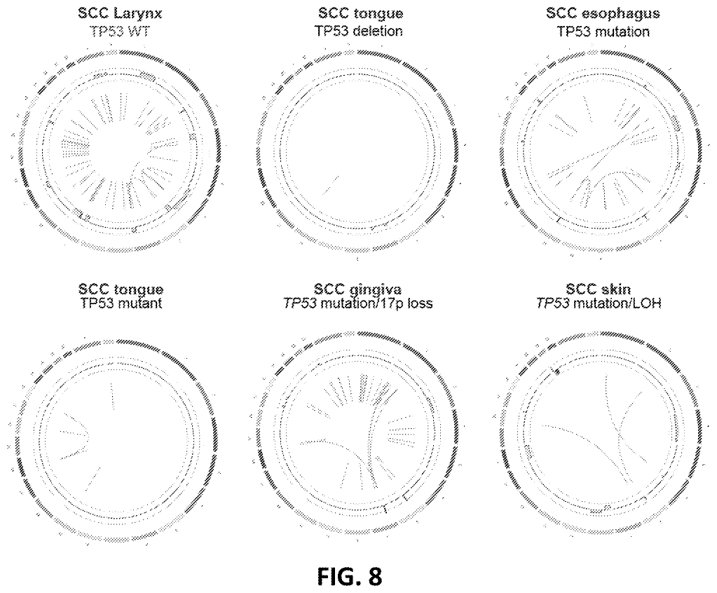

Since short telomeres have been reported to drive genome instability in model systems, the structural variant (SV) burden was analyzed, and it was found that the total number of SVs was in fact lower than sporadic head and neck SCC (FIG. 211). Moreover, using prediction algorithms which use contemporary and validated criteria that require co-occurrence of structural and copy number variants, there was no evidence of chromothripsis (FIGS. 21-2J and FIG. 8). Nonreciprocal translocations are the most common SVs associated with telomere dysfunction in yeast and mice, but these events accounted for only 16% of all the structural variants (56 of 346 events), and some tumors had no unbalanced translocations (FIG. 2K). To test if telomeres were involved in these translocations, telomere repeats were examined around the breakpoint junctions of all the SVs including those involved in non-reciprocal translocation. The mean distance of each breakpoint to the closest telomere was 27.4 MB (FIG. 2L), and there were no telomere end-to-end fusions in the unaligned whole genome sequence reads.

### Example 4—Squamous Cell Carcinomas Arise in Individuals with T Cell Immunodeficiency

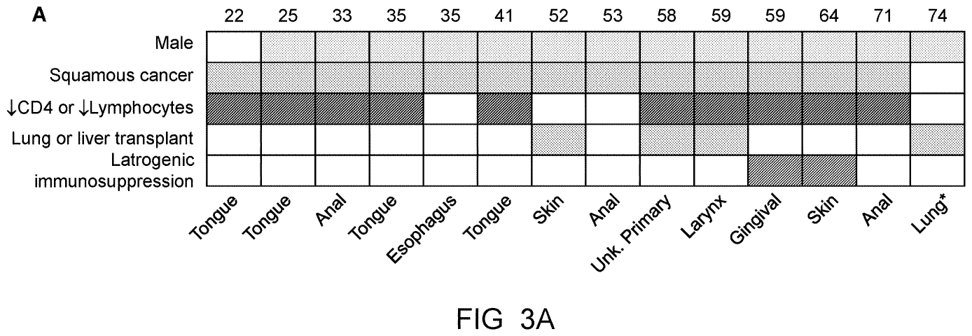

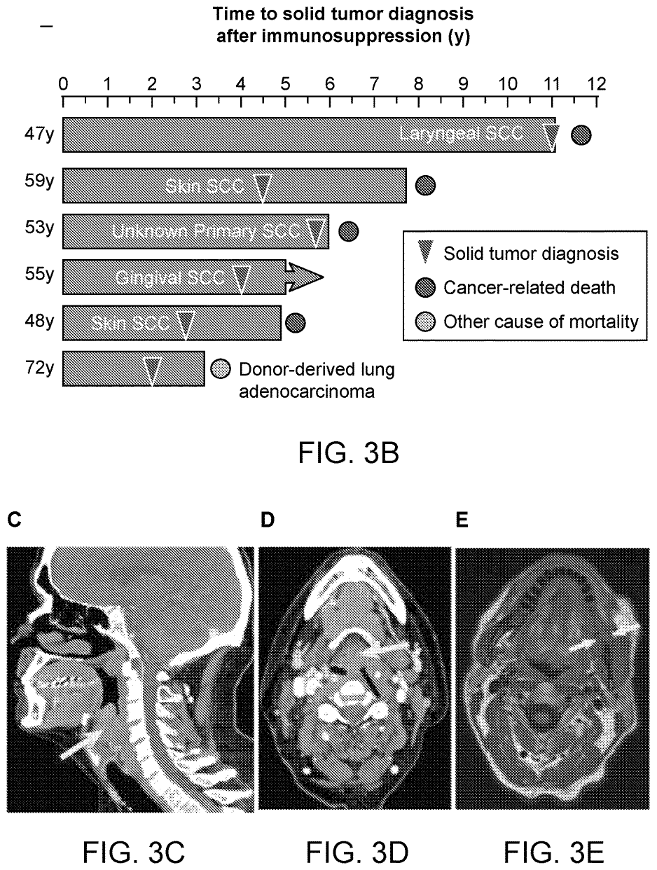

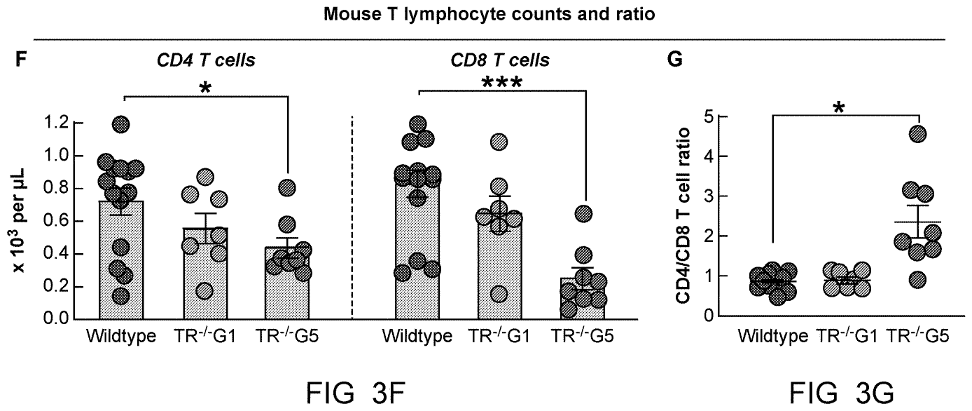

Although the squamous cancer spectrum did not overlap with the common cancers of aging, it was recognized that it included malignancies that have an increased risk in patients with T cell immunodeficiency including solid organ transplant recipients on immunosuppression and those with HIV/AIDS. It was examined and found that nearly all the patients had a T cell immunodeficiency at the time of diagnosis (86%, 12 of 14, FIG. 3A and Table 6). Individuals diagnosed prior to age 50 years had a primary unprovoked lymphopenia and severely low CD4 T cell counts (mean 305/mm³ with 5 of 6 falling below the lower end of the normal range, Table 6). Older adults had a milder baseline T cell lymphopenia, but had additionally been exposed to T cell immunosuppressive medications prior to their cancer diagnosis (Table 6). In the latter group, SCC was diagnosed after lung/liver transplantation, or after T cell cytotoxic medications (e.g. azathioprine or mycophenolate) were prescribed for presumed autoimmune disease prior to the diagnosis of a STS (FIG. 3A and Table 6). The median time to diagnosis of a solid tumor after initiation of immunosuppression was 4.25 years (range 2-11 years, n=6), and 4 of these older individuals died within one year from rapidly progressive cancer (FIGS. 3B-3E). Supporting a potential link between primary T cell immunodeficiency and risk for cancer, none of the individuals who underwent hematopoietic stem cell transplantation with T cell engraftment developed solid tumors (0 of 16, 51 lifetime years). Since T cell immunity protects against virally-induced cancers, more than 9000 viral genomes in the whole genome sequence were examined in addition to those commonly assayed in tumors clinically, and it was found that only a minority of the tumors were virally associated (4 of 14 cancers); all of them contained human papilloma virus (3 anal, 1 laryngeal SCC, FIG. 1F and Table 6).

TABLE 6

| | | | | | | | T cell immunodeficiency | |
|---|---|---|---|---|---|---|---|---|
| Age at | | | | Smoking | HPV | ANC | CD4/ALC* per mm³ | Secondary |
| diagnosis | M/F | Solid Tumor | Stage | History | Status | per mm [symbol] | [symbol] | risk factor |
| 22 | F | SCC tongue | Stage I | Never | Negative | Normal (2300) | 115/370 | No |
| 25 | M | SCC tongue | Stage I | Former | Negative | Normal (1600) | 204/— | HIV |
| 33 | M | SCC anal | Stage I | Former | Positive | — | 195/— | No |
| 36 | | SCC gingiva | Stage I | | Negative | | | |
| 35 | M | SCC tongue | Stage II | Current | Negative | Normal (2260) | 389/— | No |
| 35 | M | SCC esophagus | Stage I | Former | Negative | — | 701/— | No |
| 38 | | Anorectal | Stage IIIA | | Negative | | | |
| | | adenocarcinoma | | | | | | |
| 41 | M | SCC tongue | Stage IVA | Never | Negative | Normal (4500) | 226/— | No |
| 52 | M | SCC skin | Stage IV | Never | Negative | Normal (1890) | —/— | Lung transplant |
| 53 | M | SCC anal | Stage IIIB | Never | Positive | Normal (2040) | 735/1690^{†} | No |
| 58 | M | SCC unknown | Stage IV | Never | Negative | Normal | —/1230 | Liver transplant |
| | | primary | | | | | | |
| 59 | M | SCC larynx | Stage IVA | Never | Positive | Normal (2490) | —/790 | Lung transplant |
| 59 | M | SCC floor of | Stage IVA | Never | n/a | Normal (3700) | —/640 | Mycophenolate × 4 y |
| | | mouth | | | | | | |
| 64 | M | SCC skin | Stage II | Never | Negative | Normal (1900) | —/300 | [symbol] × 5 y |
| 71 | M | SCC anal | Stage I | Never | Positive | Normal | 337/— | No |
| 74 | M | Lung | Stage I | Never | n/a | Normal (4830) | —/1300 | Lung transplant |
| | | adenocarcinoma | | | | | | |
| | | (donor-derived) | | | | | | |

[symbol]

indicates data missing or illegible when filed

### Example 5—Mice with Short Telomeres have Defective Tumor Immune Surveillance

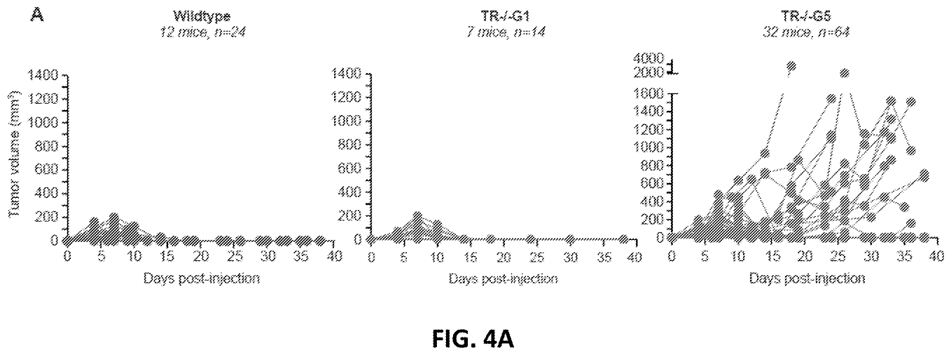

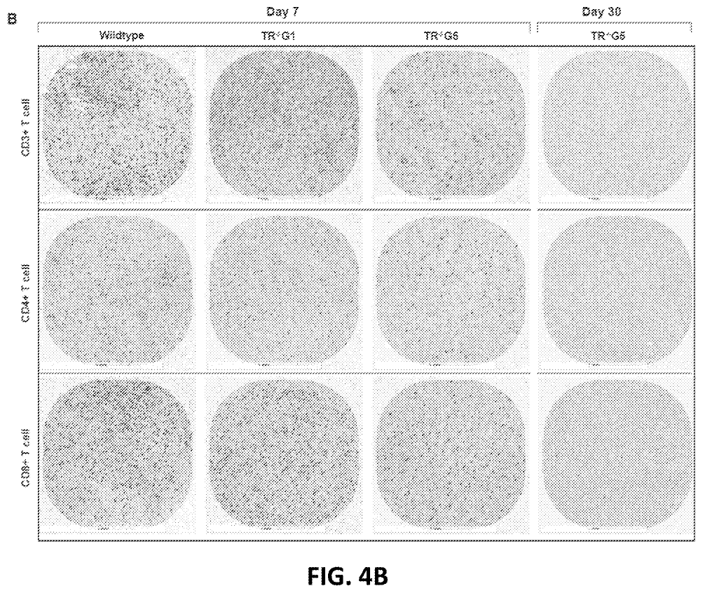

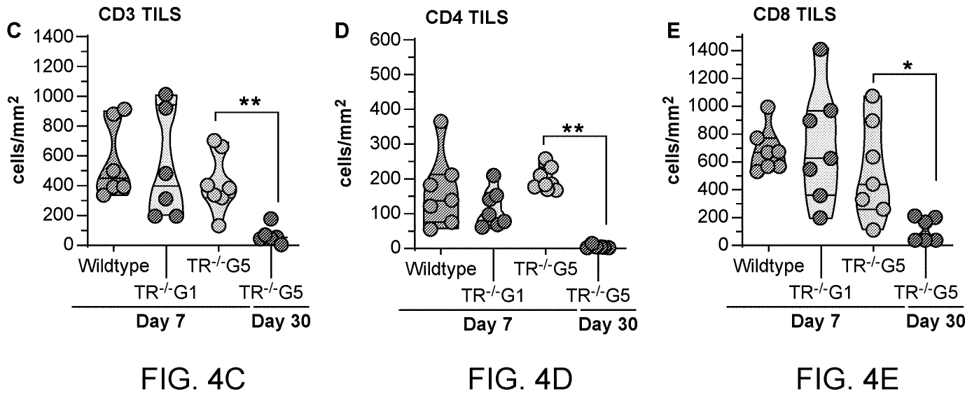

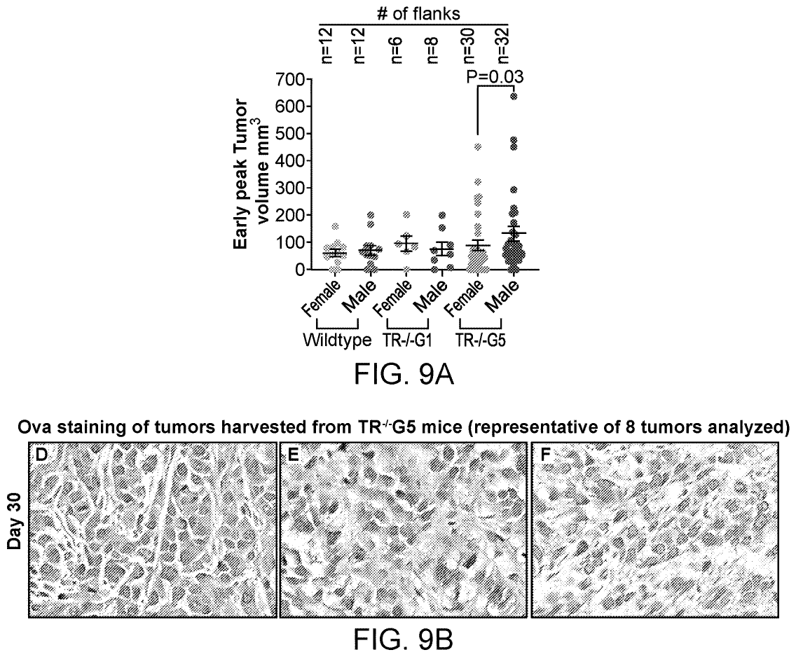

Intact replicative potential and telomere length are essential for T cell proliferation, expansion and sustained memory with aging. In contrast to children with severe STS who have a complex immunodeficiency that contributes to early mortality, adults with STS predominantly suffer from a T cell immunodeficiency of variable penetrance. The compromised T cell immunity is provoked by depleted hematopoietic stem cells and by the DNA damage response which provokes p53-dependent intrinsic apoptosis that leads to premature thymic involution. With aging, the burden of T cell apoptosis and dropout manifests as a progressive T cell lymphopenia, restriction of T cell repertoire and a propensity for opportunistic infections including reactivation of latent DNA viruses. The co-occurrence of T cell immunodeficiency with SCC led the study to hypothesize that short telomere length impairs T cell-dependent cancer immunity. To test this, telomerase RNA null mice (TR−/−) were studied, which have no phenotype in the first generation (G1), but after successive breeding, later generation mice (fifth generation. G5) acquire short telomere length. These mice recapitulate the T cell defects seen in patients with STS, including in their CD4 and CD8 lymphopenia (FIGS. 3F-3G). To test the impact of short telomere length in T cell immune surveillance capacity, immunogenic tumor cells were implanted wherein the immunogenic tumor cells were derived from KrasG12D/+Tp53R172H/+ mice that were engineered to express the human ovalbumin antigen. These cells grow tumors initially, but subsequently elicit a CD8-dependent host response that eradicates them. Although this is not a model of SCC, the effect of short telomere length on T cell cancer surveillance capacity was tested in vivo. A randomized study was performed that was frequency-matched for age and sex with a prespecified endpoint of tumor growth. Cells implanted into wildtype and TR−/−GT mice initially generated tumors, but they all became undetectable after 14 days (0 of 12 and 0 of 7 mice: 0 of 24 and 0 of 14 flanks with relapse, respectively, FIG. 4A). In contrast, 41% of TR−/−G5 mice (13 of 32) had measurable tumors (P=0.0009, Fisher's exact test, adjusted for age and sex), and 30% of implanted cells became tumorigenic (19 of 64 flanks, P<0.0001, Fisher's exact test). In a subset of cases, tumors grew unchecked after implantation (8 of 19 flanks, 42%), but more commonly, tumors were initially suppressed, similar to controls, but then relapsed (11 of 19 flanks, FIG. 4A). Male and female wildtype and TR−/−G1 mice showed comparable tumor sizes during the first 14 days, but male TR−/−G5 acquired larger tumors than females (81.8 mm³ vs. 51.8 mm³, P=0.03, Mann-Whitney test, FIG. 9A). To understand the mechanism of impaired immune surveillance, it was first assessed if TR−/−G5 tumors lost the immunogenic antigen, but it was found that ovalbumin was retained at the time of relapse (8 of 8 tumors analyzed, FIG. 9B). When analyzing the tumor infiltrating lymphocytes (TILs), it was found there was a robust CD4 and CD8 T cell infiltrate initially at day 7 across all the groups (FIGS. 4B-4E). However, by day 30, the tumor infiltrating lymphocytes had vanished in TR−/−G5 with essentially no CD4+ T cells visible and markedly fewer CD8+ T cells compared to three weeks earlier (FIGS. 4B-4E).

1. A method of treating pulmonary fibrosis or liver disease or bone marrow failure or primary immunodeficiency in a subject having short telomere syndrome (STS), the method comprising:
(a) detecting the presence of STS in the subject; and
(b) administering a therapy, wherein the therapy does not suppress T cell immunity or wherein the therapy does not lower immunosuppression.

2. The method of claim 1, further comprising:
(a) evaluating a telomere length of the subject to detect the presence of STS in the subject; and
(b) evaluating an immune status of the subject to determine whether the subject has T cell immunodeficiency.

3. The method of claim 1, further comprising detecting a gene mutation in the subject.

4. The method of claim 3, wherein the gene mutation comprises a TERT mutation, TR mutation, RTEL1 mutation, DKC1 mutation, TINF2 mutation, NAF1 mutation, PARN mutation, ZCCHC8 mutation, NHP2 mutation, NOP10 mutation, ACD mutation, STN1 mutation, CTC1 mutation, or TCAB1 mutation.

5. The method of claim 3, wherein the gene mutation comprises a loss of function mutation or a gain of function mutation.

6. The method of claim 3, wherein the gene mutation is selected from the group consisting of an insertion, a deletion, a substitution, a deletion-insertion, a duplication, an inversion, a frameshift, a repeat expansion, a translocation, and combinations thereof.

7. The method of claim 1, wherein the therapy comprises bone marrow transplant, wherein the bone marrow transplant reverses the T cell immunodeficiency.

8-14. (canceled)

15. A method of treating a squamous cell cancer, the method comprising:
(a) evaluating a telomere length of the subject to determine whether the subject has short telomere syndrome (STS);
(b) evaluating an immune status of the subject to determine whether the subject has T cell immunodeficiency;
(c) identifying the subject as having the squamous cell cancer; and
(d) administering an anti-cancer treatment to the subject.

16. The method of claim 15, further comprising detecting a gene mutation in the subject.

17. The method of claim 16, wherein the gene mutation comprises a TERT mutation, TR mutation, RTEL1 mutation, DKC1 mutation, TINF2 mutation, NAF1 mutation, PARN mutation, ZCCHC8 mutation, NHP2 mutation, NOP10 mutation, ACD mutation, STN1 mutation, CTC1 mutation, or TCAB1 mutation.

18. The method of claim 16, wherein the gene mutation comprises a loss of function mutation or a gain of function mutation.

19. The method of claim 16, wherein the gene mutation is selected from the group consisting of an insertion, a deletion, a substitution, a deletion-insertion, a duplication, an inversion, a frameshift, a repeat expansion, a translocation, and combinations thereof.

20. The method of claim 15, wherein the squamous cell cancer is a head and neck cancer, esophageal, anal cancer, or skin cancer.

21. The method of claim 15, wherein the anti-cancer treatment comprises ionizing radiation, a chemotherapeutic agent, a therapeutic antibody, a checkpoint inhibitor, or any combination thereof.

22. A method of identifying a subject as having T-cell immunodeficiency, the method comprising:
(a) evaluating telomere length of the subject to determine whether the subject has short telomere syndrome (STS); and
(b) stratifying the subject based on the subject having STS, thereby identifying the subject as having T-cell immunodeficiency.

23. The method of claim 22, further comprising detecting a gene mutation in the subject.

24. The method of claim 23, wherein the gene mutation comprises a TERT mutation, TR mutation, RTEL1 mutation, DKC1 mutation, TINF2 mutation, NAF1 mutation, PARN mutation, ZCCHC8 mutation, NHP2 mutation, NOP10 mutation, ACD mutation, STN1 mutation, CTC1 mutation, or TCAB1 mutation.

25. The method of claim 23, wherein the gene mutation comprises a loss of function mutation or a gain of function mutation.

26. The method of claim 23, wherein the gene mutation is selected from the group consisting of an insertion, a deletion, a substitution, a deletion-insertion, a duplication, an inversion, a frameshift, a repeat expansion, a translocation, and combinations thereof.
